SEQUENCE LISTING (57 sequences)

SEQ ID NO: 1 - DNA, 36 bp, Artificial Sequence
  Synthetic construct
agcttaaaaaactaccgttgttataggtgctttttg

SEQ ID NO: 2 - DNA, 36 bp, Artificial Sequence
  Synthetic construct
gatccaaaaagcacctataacaacggtagtttttta

SEQ ID NO: 3 - RNA, 25 nt, Artificial Sequence
  Synthetic construct
acuaccguuguuauaggugcuuuuu

SEQ ID NO: 4 - RNA, 25 nt, Artificial Sequence
  Synthetic construct
gcaccuauaacaacgguaguuuuuu

SEQ ID NO: 5 - RNA, 25 nt, Artificial Sequence
  Synthetic construct
gcagcacgacuucuucaagcuuuuu

SEQ ID NO: 6 - RNA, 25 nt, Artificial Sequence
  Synthetic construct
gcuugaagaagucgugcugcuuuuu

SEQ ID NO: 7 - RNA, 25 nt, Artificial Sequence
  Synthetic construct
gcuugaagaagucgugcugcuuuuu

SEQ ID NO: 8 - RNA, 25 nt, Artificial Sequence
  Synthetic construct
gcagcacgacuucuucaagcuuuuu

SEQ ID NO: 9 - RNA, 24 nt, Artificial Sequence
  Synthetic construct
gggcgagguuagacggccuuuuuu

SEQ ID NO: 10 - RNA, 24 nt, Artificial Sequence
  Synthetic construct
aggccgucuaaccucgcccuuuuu

SEQ ID NO: 11 - RNA, 24 nt, Artificial Sequence
  Synthetic construct
aggccgucuaaccucgcccuuuuu

SEQ ID NO: 12 - RNA, 24 nt, Artificial Sequence
  Synthetic construct
gggcgagguuagacggccuuuuuu

SEQ ID NO: 13 - RNA, 47 nt, Artificial Sequence
  Synthetic construct
acuaccguuguuauaggugcaagaggacucuuggacucucacuuuuu

SEQ ID NO: 14 - RNA, 47 nt, Artificial Sequence
  Synthetic construct
gugagaguccaagaguccucuugcaccuauaacaacgguaguuuuuu

SEQ ID NO: 15 - RNA, 53 nt, Artificial Sequence
  Synthetic construct
gcagcacgacuucuucaaguccgaagggcgagguuagacggccuacccuuuuu

SEQ ID NO: 16 - RNA, 53 nt, Artificial Sequence
  Synthetic construct
ggguaggccgucuaaccucgcccuucggacuugaagaagucgugcugcuuuuu

SEQ ID NO: 17 - RNA, 49 nt, Artificial Sequence
  Synthetic construct
gcagcacgacuucuucaaguccgaagggcgagguuagacggccuuuuuu

SEQ ID NO: 18 - RNA, 49 nt, Artificial Sequence
  Synthetic construct
aggccgucuaaccucgcccuucggacuugaagaagucgugcugcuuuuu

SEQ ID NO: 19 - RNA, 45 nt, Artificial Sequence
  Synthetic construct
gcagcacgacuucuucaagaagggcgagguuagacggccuuuuuu

SEQ ID NO: 20 - RNA, 45 nt, Artificial Sequence
  Synthetic construct
aggccgucuaaccucgcccuucuugaagaagucgugcugcuuuuu

SEQ ID NO: 21 - RNA, 39 nt, Artificial Sequence
  Synthetic construct
gcagcacgacuucaagggcgagguuagacggccuuuuuu

SEQ ID NO: 22 - RNA, 39 nt, Artificial Sequence
  Synthetic construct
aggccgucuaaccucgcccuugaagucgugcugcuuuuu

SEQ ID NO: 23 - RNA, 49 nt, Artificial Sequence
  Synthetic construct
gcagcacgacuucuucaaguccguuaggccgucuaaccucgcccuuuuu

SEQ ID NO: 24 - RNA, 49 nt, Artificial Sequence
  Synthetic construct
gggcgagguuagacggccuaacggacuugaagaagucgugcugcuuuuu

SEQ ID NO: 25 - RNA, 45 nt, Artificial Sequence
  Synthetic construct
gcagcacgacuucuucaaguuaggccgucuaaccucgcccuuuuu

SEQ ID NO: 26 - RNA, 45 nt, Artificial Sequence
  Synthetic construct
gggcgagguuagacggccuaacuugaagaagucgugcugcuuuuu

SEQ ID NO: 27 - RNA, 39 nt, Artificial Sequence
  Synthetic construct
gcagcacgacuucuuaggccgucuaaccucgcccuuuuu

SEQ ID NO: 28 - RNA, 39 nt, Artificial Sequence
  Synthetic construct
gggcgagguuagacggccuaagaagucgugcugcuuuuu

SEQ ID NO: 29 - RNA, 24 nt, Artificial Sequence
  Synthetic construct
gucucguagaccgugcaccuuuuu

SEQ ID NO: 30 - RNA, 24 nt, Artificial Sequence
  Synthetic construct
ggugcacggucuacgagacuuuuu

SEQ ID NO: 31 - RNA, 24 nt, Artificial Sequence
  Synthetic construct
ggcacaugguaucgacccuuuuuu

SEQ ID NO: 32 - RNA, 24 nt, Artificial Sequence
  Synthetic construct
agggucgauaccaugugccuuuuu

SEQ ID NO: 33 - RNA, 41 nt, Artificial Sequence
  Synthetic construct
gucucguagaccgugaaggcacaugguaucgacccuuuuuu

SEQ ID NO: 34 - RNA, 41 nt, Artificial Sequence
  Synthetic construct
agggucgauaccaugugccuucacggucuacgagacuuuuu

SEQ ID NO: 35 - RNA, 41 nt, Artificial Sequence
  Synthetic construct
gucucguagaccguguuagggucgauaccaugugccuuuuu

SEQ ID NO: 36 - RNA, 41 nt, Artificial Sequence
  Synthetic construct
ggcacaugguaucgacccuaacacggucuacgagacuuuuu

SEQ ID NO: 37 - RNA, 45 nt, Artificial Sequence
  Synthetic construct
gucucguagaccgugcaccaaggcacaugguaucgacccuuuuuu

SEQ ID NO: 38 - RNA, 45 nt, Artificial Sequence
  Synthetic construct
agggucgauaccaugugccuuggugcacggucuacgagacuuuuu

SEQ ID NO: 39 - RNA, 45 nt, Artificial Sequence
  Synthetic construct
gucucguagaccgugcaccuuagggucgauaccaugugccuuuuu

SEQ ID NO: 40 - RNA, 45 nt, Artificial Sequence
  Synthetic construct
ggcacaugguaucgacccuaaggugcacggucuacgagacuuuuu

SEQ ID NO: 41 - RNA, 21 nt, Artificial Sequence
  Synthetic construct
aagucucguagaccgugcacc

SEQ ID NO: 42 - RNA, 21 nt, Artificial Sequence
  Synthetic construct
aaggcacaugguaucgacccu

SEQ ID NO: 43 - DNA, 22 bp, Artificial Sequence
  Synthetic construct
cggaattccccagtggaaagac

SEQ ID NO: 44 - DNA, 23 bp, Artificial Sequence
  Synthetic construct
cggaattcatatttgcatgtcgc

SEQ ID NO: 45 - DNA, 19 bp, Artificial Sequence
  Synthetic construct
gcagcacgacttcttcaag

SEQ ID NO: 46 - DNA, 19 bp, Artificial Sequence
  Synthetic construct
gggcgaggttagacggcct

SEQ ID NO: 47 - DNA, 26 bp, Artificial Sequence
  Synthetic construct
tagtatatgtgctgccgaagcgagca

SEQ ID NO: 48 - DNA, 20 bp, Artificial Sequence
  Synthetic construct
actaggccgagagccacggg

SEQ ID NO: 49 - DNA, 21 bp, Artificial Sequence
  Synthetic construct
ttggtccaggactgtgccgat

SEQ ID NO: 50 - DNA, 20 bp, Artificial Sequence
  Synthetic construct
tccacgaggttgcgaccgct

SEQ ID NO: 51 - DNA, 20 bp, Artificial Sequence
  Synthetic construct
gtcagttgagtggcactcat

SEQ ID NO: 52 - DNA, 20 bp, Artificial Sequence
  Synthetic construct
atgaccaacaagtgtctcct

SEQ ID NO: 53 - DNA, 20 bp, Artificial Sequence
  Synthetic construct
tcagtttcggaggtaacctg

SEQ ID NO: 54 - DNA, 21 bp, Artificial Sequence
  Synthetic construct
tcagaagagaagccaacgtga

SEQ ID NO: 55 - DNA, 20 bp, Artificial Sequence
  Synthetic construct
cggagacagcgagggtaaat

SEQ ID NO: 56 - DNA, 22 bp, Artificial Sequence
  Synthetic construct
agtatggtgtcgacataccgga

SEQ ID NO: 57 - DNA, 22 bp, Artificial Sequence
  Synthetic construct
gagtctggtaaacagccgaatg

(19) United States
(12) Patent Application Publication
(10) Pub. No.: US 2011/0008885 A1
(43) Pub. Date: Jan. 13, 2011

(54) LINEAR DOUBLE-STRANDED RNA MOLECULE INTERFERING WITH DIFFERENT TARGET GENES

(75) Inventors: Meehyein Kim, Yongin-si (KR); Duckhyang Shin, Yongin-si (KR); Hyeon Lee, Yongin-si (KR); Soo In Kim, Yongin-si (KR); Yeup Yoon, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Mogam Biotechnology Research Institute, Yongin-si, Gyeonggi-do (KR)

(21) Appl. No.: 12/667,738

(22) PCT Filed: Jul. 7, 2008
(86) PCT No.: PCT/KR2008/003982

§ 371 Date: Jan. 5, 2010

Related U.S. Application Data
(60) Provisional application No. 60/948,256, filed on Jul. 6, 2007.

(51) Int. Cl. C12N 5/071; C07H 21/02; C12N 15/63

(52) U.S. Cl. 435/366; 536/24.5; 435/320.1

(57) ABSTRACT

A linear double-stranded RNA molecule, which comprises two or more consecutively or convergently linked short interfering RNAs (siRNAs) each reducing the expression of one of different target genes, and a recombinant expression vector comprising double-stranded DNA sequence expressing the linear double-stranded RNA molecule are provided. The linear double-stranded RNA molecule or the recombinant expression vector is useful for a method of reducing expression of target genes in a cell, the method comprising introducing the linear double-stranded RNA molecule or the recombinant expression vector into the cell, whereby the encoded siRNAs target different genes and reduce expression of the target genes. It was also proved that effective gene silencing activity can be induced when each siRNA unit within the linear double-stranded RNA molecule has 18 to 24 nucleotides and, additionally, the gene silencing activity is not affected by inverted orientation of an siRNA.

### FIELD OF THE INVENTION

The present invention relates to a linear double-stranded RNA molecule interfering with different target genes, a recombinant expression vector therefor and a method of reducing expression of target genes in a cell employing the linear double-stranded RNA molecule or the recombinant expression vector.

### BACKGROUND OF THE INVENTION

Hepatitis C virus (HCV) belongs to Flaviviridae family and has an approximately 9.6 kb positive strand RNA genome flanked with 5′- and 3′-untranslated region (UTR), which encodes at least 10 viral structural and nonstructural proteins (Grakoui, A. et al., 1993, J Virol 67:1385-1395; Bartenschlager, R. et al., 2000, J Gen Virol 81:1631-1648). An estimated 170 million people worldwide are infected with this virus chronically. It has also been known as a major agent causing hepatitis, liver cirrhosis and hepatocellular carcinoma (Alter, M. J. et al., 1997, Hepatology 26:62 S-65S). Unfortunately, neither prophylactic nor therapeutic vaccine against HCV was yet commercially available. Although the combination therapy with interferon (IFN)-α and ribavirin has resulted in remarkable outcomes in clinical applications, about half of the HCV-infected patients is benefiting by this treatment (Chander, G. et al., 2002, Hepatology 36:S135-144). Thus, there is an urgent need for developing an alternative therapeutics to control HCV infection.

RNA interference (RNAi) is a post-transcriptional gene silencing process and evolutionally conserved in plants, Caenorhabditis elegans and animals (Bosher, J. M. et al., 2000, Nat Cell Biol 2:E31-36; Dykxhoorn, D. M. et al., 2003, Nat Rev Mol Cell Biol 4:457-467). In mammalian cells, RNase III-like ribonuclease named Dicer recognizes long double-stranded RNA (dsRNA) and chops it into shorter duplex RNAs, small interfering RNAs (siRNA), of 21-25 nucleotides (nt) in length in the cellular cytoplasm (Dorsett, Y. et al., 2004, Nat Rev Drug Discov 3:318-329). Previous studies have shown that the RNAi-based technology has great promise particularly for the treatment of RNA virus-derived diseases, the RNA virus including the human immunodeficiency virus type 1 (HIV-1) and HCV (Capodici, J. et al., 2002, J Immunol 169:5196-5201; Kapadia, S. B. et al., 2003, Proc Natl Acad Sci USA 100:2014-2018; Kim, M. et al., 2006, Virus Res 122:1-10). As the viral RNA transcripts are produced or replicated in the cytoplasm, where cellular metabolic process can also initiate the RNAi machinery, these viruses have been expected to be efficiently controlled by siRNA-mediated therapy. However, the mutation rate of these viruses is high due to a weak, non-stringent proof-reading activity of viral RNA polymerases, resulting in rapid emergency of escape variants from RNAi (Das, A. T. et al., 2004, J Virol 78:2601-2605; Wilson, J. A. et al., 2005, J Virol 79:7050-7058).

Specifically, it has been suggested in recent reports that this problem could be resolved by the use of synthetic siRNA mixture with several sequences, in vitro Dicer-generated siRNA products from long dsRNA, or intracelluary-expressed long hairpin RNA (lhRNA) (Wilson, J. A. et al., 2005, J Virol 79:7050-7058; Watanabe, T. et al., 2006, Gene Ther 13:883-892; Liu, Y. P. et al., 2007, Nucleic Acids Res 35:5683-5693; Sano, M. et al., 2008, Mol Ther 16:170-177). Plasmids encoding lhRNA from an RNA Pol III promoter were constructed either by successive extension of an shRNA target sequence or constitutive combination of multiple targets. They showed efficient inhibition of expression or replication of human pathogenic viruses, including HIV-1, HBV and HCV, and also reduction of chance of viral escape. Especially, in the latter multi-targeting approach, two functional siRNAs against HIV-1 were processed from an expressed lhRNA and then silenced separated genes, pol and nef, in cells. Moreover, as distinct advantages, insertion of G:U wobble pairs in the hairpin stem and removal of perfect complementarity within the inverted repeat sequences not only facilitated cloning/sequencing but also avoided non-specific IFN response. However, the present inventors observed that the RNAi activity was affected by the content of G:U base pairs even in shRNA and its acceptable number was depending on the target sequences, indicating that maintenance of RNAi activity of individual shRNA components with G:U wobbles should be confirmed prior to their combination into a single expression cassette. Another potential concern of using lhRNA is that dicer-mediated cleavage is progressed from the base to the loop of the hairpin sequentially, causing unequal production yield of different kinds of siRNA, due to saturation or reduction of enzymatic activity of dicer, and thus insufficient multi-silencing effects presumably.

### SUMMARY OF THE INVENTION

Accordingly, it is an object of the present invention to provide an RNA molecule producing different kinds of siRNA in equal yields.

It is another object of the present invention to provide an expression vector for producing said RNA molecule.

It is a further object of the present invention to provide a composition comprising said RNA molecule or an expression vector therefor.

It is a still further object of the present invention to provide a method of reducing expression of target genes in a cell by employing said RNA molecule or the recombinant expression vector.

In accordance with one aspect of the present invention, there is provided a linear double-stranded RNA (liRNA) molecule which comprises two or more consecutively or convergently linked siRNAs each reducing the expression of one of different target genes.

The present invention further provides a recombinant expression vector comprising double-stranded DNA sequences expressing the liRNA molecule.

The present invention further provides a composition comprising the liRNA molecule or the recombinant expression vector.

The present invention further provides a method of reducing expression of target genes in a cell, the method comprising introducing the liRNA molecule or the recombinant expression vector into the cell, wherein the encoded siRNAs target different genes and reduce expression of the target genes.

### BRIEF DESCRIPTION OF THE DRAWINGS

The above and other objects and features of the present invention will become apparent from the following description of the invention, when taken in conjunction with the accompanying drawings, which respectively show:

FIG. 1A to FIG. 1D: Transcription of duplex RNAs from convergently opposing Pol III promoters and their gene silencing in cells. FIG. 1A depicts the construct of a control duplex siRNA-expression plasmid, pGD-siC, comprising double-stranded DNA sequence (SEQ ID NOs: 1 and 2) expressing a control siRNA. Hybridized synthetic oligonucleotides can be inserted within HindIII and BamHI sites of the vector. RNAs with poly(U) at 3′ terminus are synthesized from the human H1 and U6 promoters. +1, the transcription initiation site. FIG. 1B shows RNA sequences of siRNAs (SEQ ID NOs: 3 to 12) and liRNAs (SEQ ID NOs: 13 to 16). siC, siE and siR indicate control, enhanced green fluorescence protein (EGFP)-specific and renilla luciferase-specific siRNAs, respectively. s and a, sense and antisense strand transcripts from the H1 promoter, respectively. liC and liER indicate long interference RNAs, which are irrelevant with or targets both EGFP and luciferase. Twenty one (21) or twenty five (25) is number of nucleotides complementary to the target mRNA. Gray boxes show the position of the antisense sequences within the duplex RNA. FIG. 1C presents RNAi activity of siE (s21), siE (a21), siR (s21), siR (a21)-expression plasmids relative to that of the control siRNA, siC, measured by dual luciferase (FLuc and RLuc) assay. FIG. 1D displays relative RNAi activity of liER (s25s25) compared with irrelevant liRNA, siC. All values were measured in triplicates.

FIG. 2A to FIG. 2C: RNAi activity of liRNAs with two siRNA components in a successive alignment. FIG. 2A shows the sequences of liER RNAs (SEQ ID NOs: 17 to 22) with different lengths targeting both EGFP and RLuc. Their sense strands are transcribed from the H1 promoter. Gray boxes show the position of the antisense sequences within the long interference RNA. Probe sequences of siE and siR probes for Northern blotting below were also indicated. FIG. 2B depicts multiple gene knockdown activity of the liRNAs measured by dual luciferase assay. Luciferase expression level by plasmid encoding control liRNA, liC, was set at 100%. Assays were performed in triplicate. FIG. 2C presents Northern blot analysis showing the efficiency and accuracy of siRNA processing from a longer RNA substrate in Huh 7 cells. The products processed into 21-23 nt siRNA were indicated with arrows. Probe sequence for U6 small nuclear RNA was hybridized as a loading control.

FIG. 3A to FIG. 3C: RNAi activity of liRNAs with two siRNA components in a convergent alignment. FIG. 1A shows sequences of liER RNAs (SEQ ID NOs: 23 to 28) with different lengths targeting both EGFP and RLuc. The sense strands of EGFP and RLuc siRNAs are transcribed from the H1 and U6 promoters, respectively. Gray boxes show the position of the antisense sequences within the long interference RNA. Probe sequences of siE and siR probes, for Northern blotting below are also indicated. FIG. 3B presents multiple gene knockdown activity of the liRNAs assessed by dual luciferase assay. Luciferase expression level by plasmid encoding control liRNA, liC, was set at 100%. Assays were performed in triplicate. FIG. 3C displays the result of Northern blot analysis showing the efficiency and accuracy of siRNA processing from a longer RNA substrate in Huh 7 cells. The products processed into 21-23 nt siRNA were indicated with arrows. Probe sequence for U6 small nuclear RNA was hybridized as a loading control.

FIG. 4A to FIG. 4C: Antiviral activity of liRNAs in HCV replicon cells, FK-R2AN. FIG. 4A shows sequences of siRNAs (SEQ ID NOs: 29 to 32) and liRNAs (SEQ ID NOs: 33 to 40) with various lengths targeting HCV core and NS3. Gray boxes show the position of the antisense sequences within the long interference RNA. FIG. 4B presents HCV RNA replication effeciency measured by renilla luciferase assay. Luciferase expression level by plasmid encoding control liRNA, liC, was set at 100%. Assays were performed in triplicate. FIG. 4C displays the result of Western blot analysis for detecting HCV core expression in replicon cells transfected with different siRNA- or liRNA-expression vectors. β-actin was used as an internal loading control.

FIG. 5A to FIG. 5C: Rapid amplification of cDNA ends (RACE) for determination of cleavage sites within the HCV replicon RNA by liRNA. FIG. 5A is a schematic presentation of the RACE experiment determining 5′ ends of liRNA cleavage sites. Arrows of siHCV-U and siHCV-N indicate their target sites in the replicon RNA genome. FIG. 5B depicts the result of PCR amplification with 5′ UTR (upper) and NS3 (middle) specific primers after reverse transcription of RACE products with random oligomers. Amplification of the β-actin gene was used as an internal control. FIG. 5C shows the result of sequence analysis of RT-PCR products from RACE. Gray bars indicate cDNA of viral RNA sequences linked to an RNA adaptor at their 5′ ends. Dotted and solid arrows respectively represent predicted and actual sites in HCV target sequences (SEQ ID NOs: 41 and 42) cleaved by siRNAs (siHCV-U and siHCV-N) or liRNAs (liHCV-UNs).

FIG. 6 shows the result of semi-quantitative RT-PCR of IFN-β, OAS1 and MxA in FK/R2AN cells transfected with indicated plasmids expressing siRNA or liRNA for confirming non-specific interferon response to liRNAs in the cells. Poly (I:C) was used as an positive control. β-actin was used as an internal loading control.

### DETAILED DESCRIPTION OF THE INVENTION

The terms used for describing the present invention are defined as follows.

A “small interfering RNA” or “short interfering RNA” or siRNA is a RNA duplex of nucleotides that targets a gene of interest (a “target gene” or a “target coding sequence”). An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene.

The term “regulatory elements” used herein refer to transcriptional and translational control sequences, such as promoters, enhancers, polyadenylation signals, terminators, protein degradation signals, and the like, that provide for and/or regulate expression of a coding sequence in a cell.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”. In the present specification, “plasmid” and “vector” are used interchangeably unless otherwise clear from the context.

The term “expression” with respect to a gene sequence refers to transcription of the gene and, as appropriate, translation of the resulting mRNA transcript to a protein.

“Inhibition of gene expression” refers to the absence (or observable decrease) in the level of protein and/or mRNA product from a target gene.

The phrase “inhibiting expression of a cellular gene by the siRNA” refers to sequence-specific inhibition of genetic expression by a small interfering RNA molecule (siRNA) characterized by degradation of specific mRNA(s). The process is also referred to as RNA interference or RNAi.

Promoters, terminators and control elements “operably linked” to a nucleic acid sequence of interest are capable of effecting the expression of the nucleic acid sequence of interest. The control elements need not be contiguous with the coding sequence, so long as they function to direct the expression thereof. Thus, for example, a promoter or terminator is “operably linked” to a coding sequence if it affects the transcription of the coding sequence. A “promoter” refers to an array of nucleic acid control sequences that direct transcription of a nucleic acid. The term “promoter” includes those promoter elements which are sufficient to render promoter-dependent gene expression controllable for cell type-specific, tissue-specific or inducible by external signals or agents. Thus, as used herein the term “promoter” is used interchangeably with the term “regulatory element(s).”

Unless defined otherwise, all technical and scientific terms used herein have the same meaning as commonly understood by one of ordinary skill in the art to which this invention belongs.

In the present invention, there is provided a linear double-stranded RNA (liRNA) molecule which comprises two or more consecutively or convergently linked short interfering RNAs (siRNAs) each reducing the expression of one of different target genes.

The liRNA molecule may comprise two to ten, preferably, two to three siRNAs, as necessary. Each of the siRNAs contained in the liRNA may comprise 18 to 24 nucleotides, preferably, about 21 nucleotides. More Preferably, the liRNA molecule may consist of two consecutive or convergent siRNAs, 40 nucleotides of one strand of the liRNA molecule being base-paired with the complementary 40 nucleotides of the other strand and each strand comprising two 3′ terminal nucleotides that are not base-paired.

The target genes of the liRNA molecule may be a gene derived from a cell, an endogenous gene, a pathologically mutated gene, e.g. a cancer causing gene, one or more genes whose expression causes or is related to heart disease, lung disease, Alzheimer's disease, Parkinson's disease, diabetes, arthritis, etc.; a transgene; or a gene of a pathogen which is present in the cell after infection thereof, e.g., a viral or bacterial pathogen. Exemplary viral genes include genes of Human Immunodeficiency Virus (HIV), Hepatitis B Virus (HBV), Hepatitis C Virus (HCV), Herpes-simplex 1 and 2, Varicella Zoster, and Rhinovirus.

In some embodiments, the liRNA molecule is composed of a set of nucleotide sequences of SEQ ID NOs: 37 and 38 or SEQ ID NOs: 39 and 40, where the target sequences are sequences derived from HCV 5′ UTR and NS3 genes, respectively.

However, given the guidance provided in the instant specification, those skilled in the art can readily generate other siRNA-encoding liRNA molecules that function to reduce expression of any of a wide variety of target genes, in accordance with the methods well-known in the art.

The inventive liRNA molecule may be used as a therapeutic agent for treating a disease caused by malfunction of an endogenous gene or infectious pathogens in a cell or an organism. Further, the liRNA molecule may be used for functional analysis of any target genes.

The present invention further provides a recombinant expression vector comprising double-stranded DNA sequences complementary to the inventive liRNA molecule. When the recombinant expression vector is introduced into a cell, efficient transcription of a primary long interfering RNA (liRNA) occurs and the liRNA molecule is processed by the cellular RNAi machinery into self-annealed siRNA molecules targeting different target sequences. Beneficially, the inventive recombinant expression vector increases success rate of sequencing reaction and genetic stability, compared to a hairpin RNA-expression construct with inverted repeats in one plasmid backbone.

The recombinant expression vector preferably comprises two convergent promoters operably linked to both ends of the double-stranded DNA, each promoter allowing transcription of each strand of the double-stranded DNA sequence.

The promoters may be human RNA polymerase III promoters, each of which is contiguous with each of 5′ ends of the double-stranded DNA. Exemplary human RNA polymerase III promoters include human H1, U6, 5S rRNA, 7SK and tRNA promoters, wherein human H1 and U6 promoters are preferred.

The human RNA Polymerase III promoter is in some embodiments a “wild-type,” or “naturally-occurring promoter. In other embodiments, the human RNA Polymerase III promoter may contain one or more differences in nucleotide sequence compared to a naturally-occurring promoter. In some embodiments, the human RNA Polymerase III promoter is a synthetic promoter, e.g., the promoter is synthesized using standard recombinant and/or synthetic methods.

The expression vector may further comprises other regulatory elements such as enhancers, polyadenylation signals, terminators, and the like, for regulating the expression of cDNA of the long linear RNA molecule.

The present invention further provides a composition comprising the inventive liRNA or the inventive recombinant expression vector. The inventive composition may be used as a pharmaceutical composition for treating a disease caused by malfunction of an endogenous gene or infectious pathogens in a cell or an organism.

Moreover, the present invention provides a method of reducing expression of target genes in a cell, the method comprising introducing the inventive liRNA molecule or the inventive recombinant expression vector into the cell, wherein the encoded siRNAs target different genes and reduce expression of the target genes.

In some embodiments, the cell is a eukaryotic cell. In some embodiments, the cell is an in vitro cell (e.g., a eukaryotic cell grown in single cell suspension or as a cell layer in vitro). In some embodiments, the target cell is an in vivo cell (e.g., a eukaryotic cell that is part of a multicellular organism).

In some embodiments, the target gene is an endogenous gene. In some embodiments, the target gene is an exogenous gene. In some embodiments, the target gene is a gene of an intracellular pathogen. In some embodiments, the target gene is a viral gene.

By reducing expression is meant that the level of expression of a target gene is reduced or inhibited by at least about 10% or more, preferably, at least about 20% or more, as compared to a non-treated control. In certain embodiments, the expression of the target gene is reduced to such an extent that expression of the target gene is effectively inhibited, such that expression is undetectable.

The consequences of inhibition can be confirmed by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucuronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof.

The inventive method may be used for treating various disorders, the methods generally involving administering the inventive liRNA molecule or the inventive recombinant expression vector to an individual, such that the RNA molecule or the expression vector enters a cell of the individual, the siRNAs encoded by the expression vector is produced in a cell, and the siRNA reduces expression of the target genes in the cell.

Depending on the nature of the condition being treated, the target gene may be a gene derived from the cell, an endogenous gene, a pathologically mutated gene, e.g. a cancer causing gene, one or more genes whose expression causes or is related to heart disease, lung disease, Alzheimer's disease, Parkinson's disease, diabetes, arthritis, etc.; a transgene; or a gene of a pathogen which is present in the cell after infection thereof, e.g., a viral or bacterial pathogen.

The inventive liRNA molecule or the inventive recombinant expression vector (the “active agent”) can be introduced into a cell using any convenient protocol, where a number of different such protocols are known in the art. The active agent may be introduced into tissues or host cells by any number of routes, including viral infection, microinjection, or fusion of vesicles. Jet injection may also be used for intra-muscular administration, as described by Furth et al. (Anal Biochem 205:365-368 (1992)). Physical methods of introducing the active agent molecule include injection directly into the cell or extracellular injection into the organism of an RNA solution. Depending on the nature of the active agent, the active agent(s) may be administered to the host using any convenient means capable of resulting in the desired modulation of target gene expression. Thus, the active agent can be incorporated into a variety of formulations for therapeutic administration. More particularly, the active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intramuscular, intratumoral, subcutaneous, intraocular, intradermal, transdermal, intracheal, etc., administration.

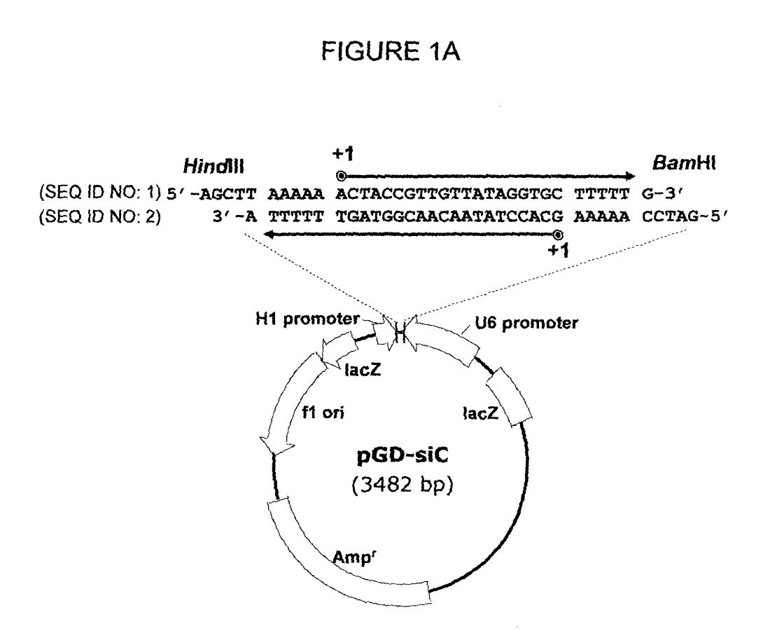

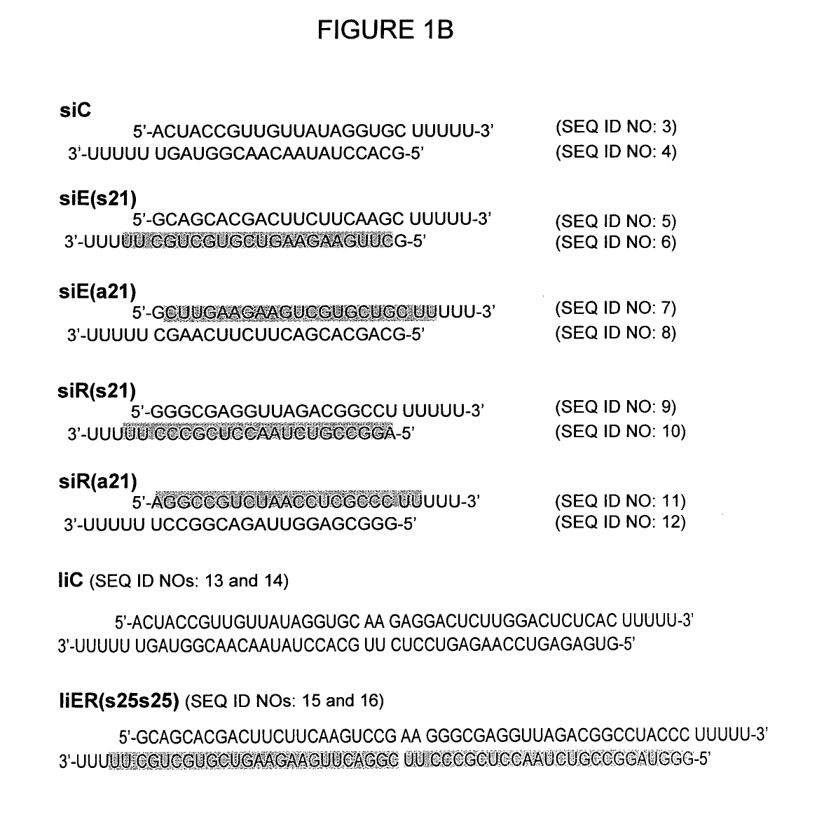

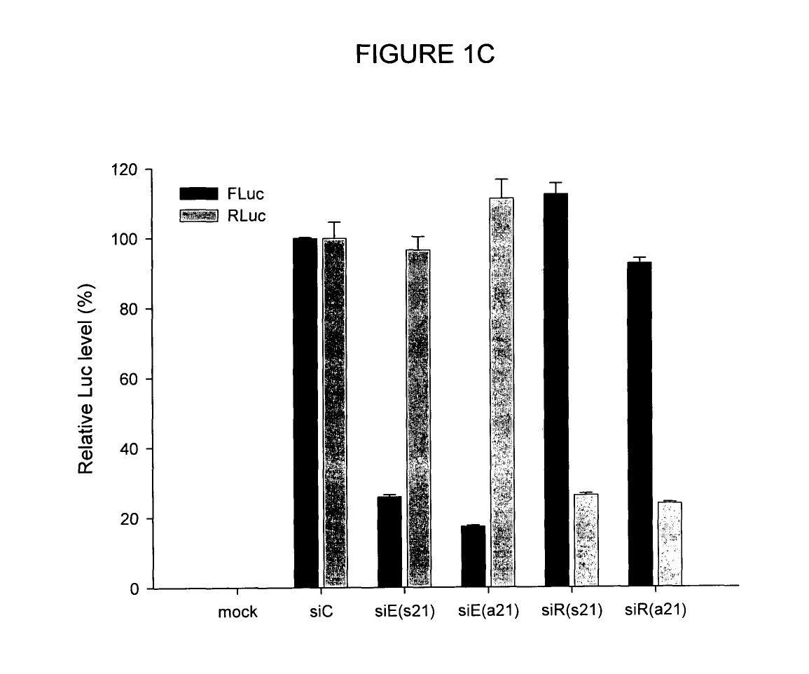

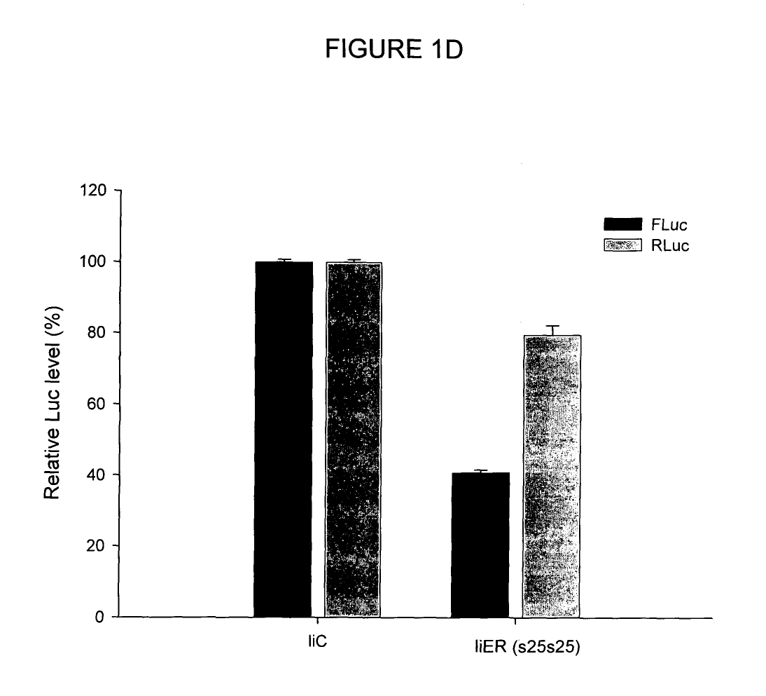

In one embodiment of the present invention, the present inventors attempted to learn the rules for designing linear duplex RNA first by targeting two different sites of EGFP and RLuc reporter genes, in a trans mRNA expression system. In the inventive liRNA vector construct (see FIG. 1A), two complementary RNA strands are transcribed separately from the convergent H1 and U6 RNA Pol III promoters and then self-annealed into a single RNA molecule. Without addition of G:U wobble pairs which was required in long hairpin RNA, there was no problem in plasmid DNA amplification in E. coli and sequencing analysis.

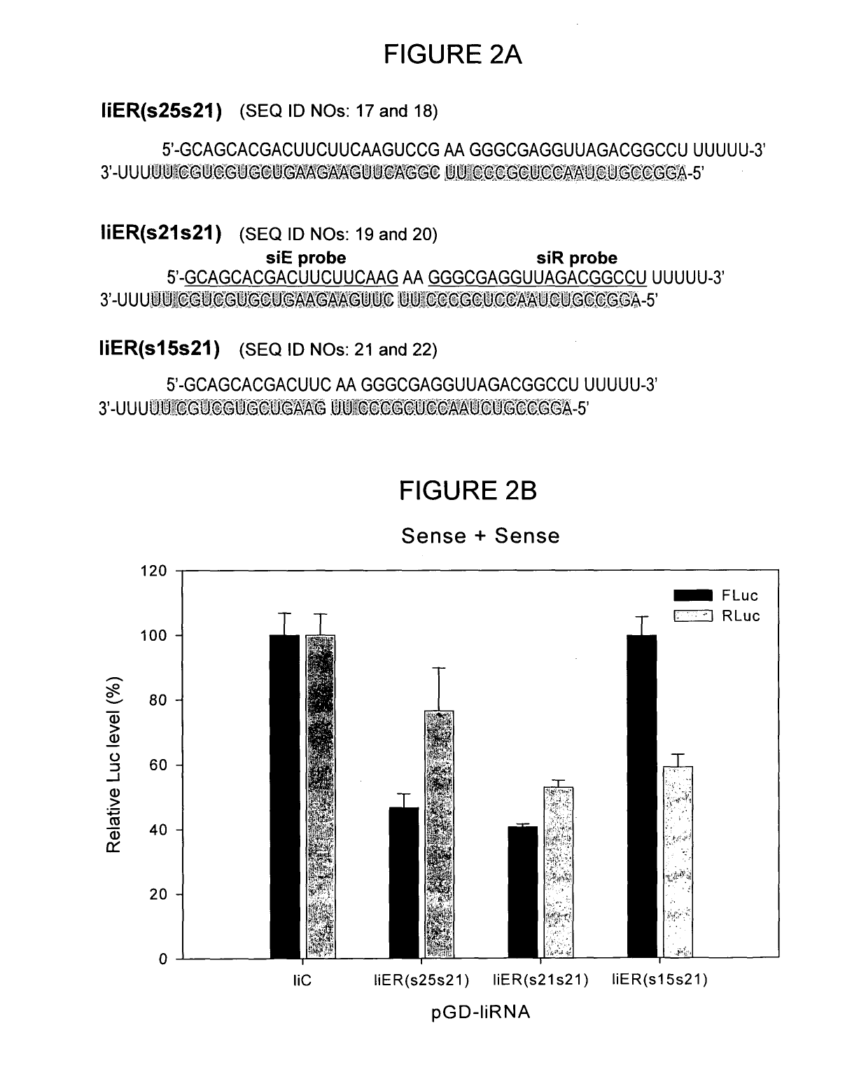

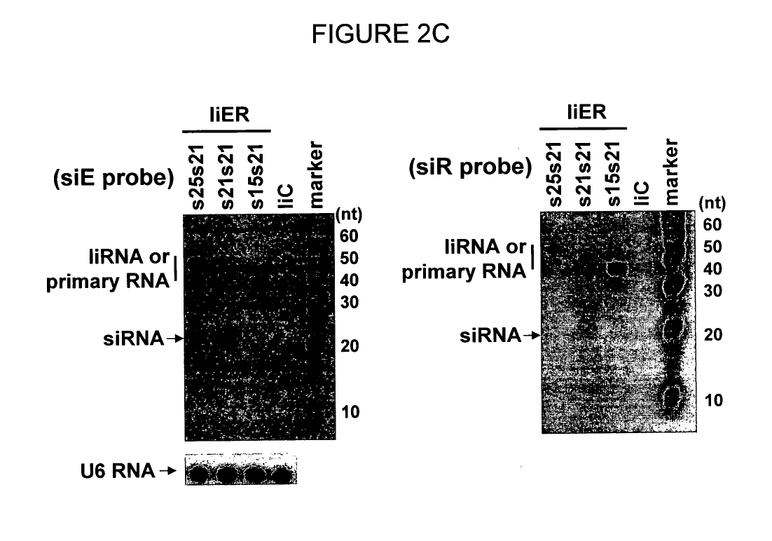

Notably, from experiments to address minimal length requirement for inducing RNAi in mammalian cells, the most appreciable knockdown was detected by linear dsRNA (21 nt+21 nt) with 40 by stem and 3′ overhangs (see FIG. 2B). However, multi-silencing efficiency was dramatically reduced when longer (44 by stem) or shorter (34 by stem) duplex RNA-expressing plasmid was transfected (see FIG. 2B). These results were correlated with the siRNA Northern blot analysis (see FIG. 2C). In contrast to this observation, a previous report (Liu Y. P. et al., Nucleic Acids Research 35:5683-5693 (2007)) on requirement of optimal length of lhRNA suggested that hairpins with 43 or 44 by stem, which contains extra sequences between the two target sequences and at the end of the stem adjacent to the loop, were more functional rather than 40 or 41 by stem-bearing cognates. This discrepancy of optimal duplex length between lhRNA and liRNA is likely to be caused by different siRNA processing mechanisms: uni-directional chopping by dicer from the base of the lhRNA stem, whereas potentially both uni- and bi-directional processing from the two exposed stem-termini of the liRNA.

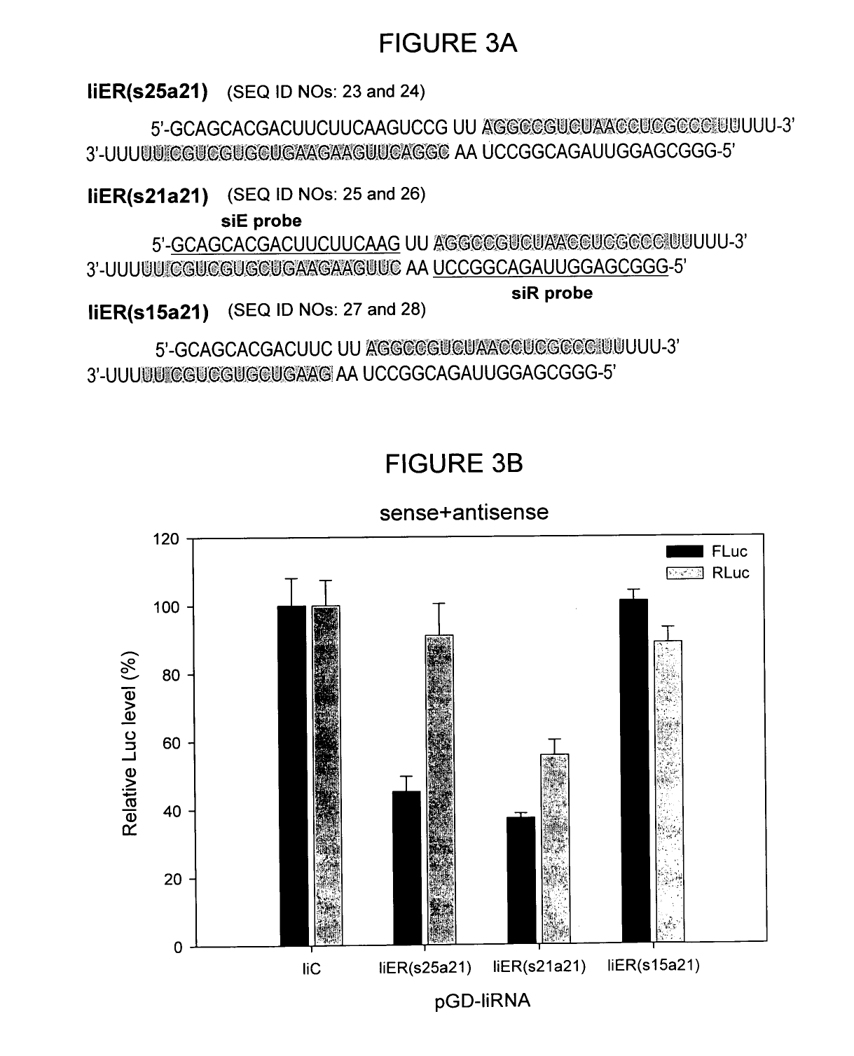

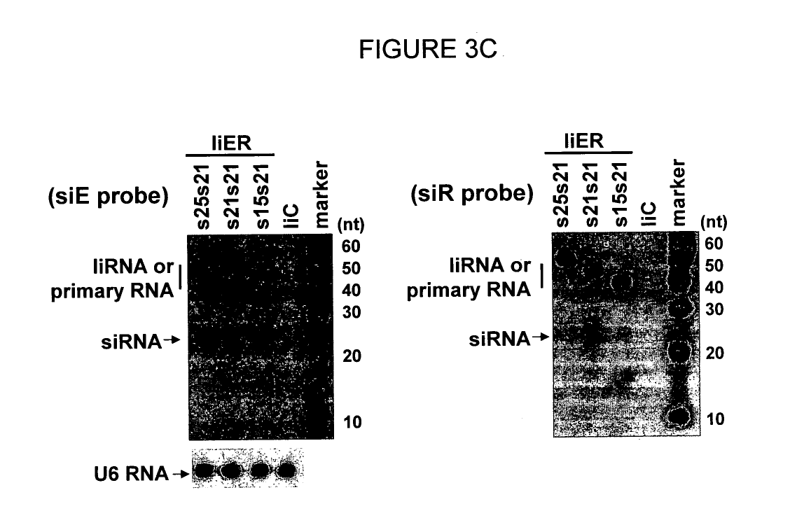

Additionally, strand selectivity was investigated by converting the orientation of RLuc siRNA unit within the liER RNA, rendering each anti-sense strand of EGFP and RLuc sequences to have 3′ overhang structure similar to the feature of the stem base of shRNA. Any liRNA with structure of either sense+sense or sense+antisense under the H1 promoter showed similar gene silencing effects (see FIG. 2B and FIG. 3B), reflecting comparable thermodynamic stability between the terminal base pairs of the RLuc siRNA. This finding confers that incorporation efficiency of guide/antisense strand into the RISC could be coordinated artificially in an optimal alignment of siRNAs within an liRNA.

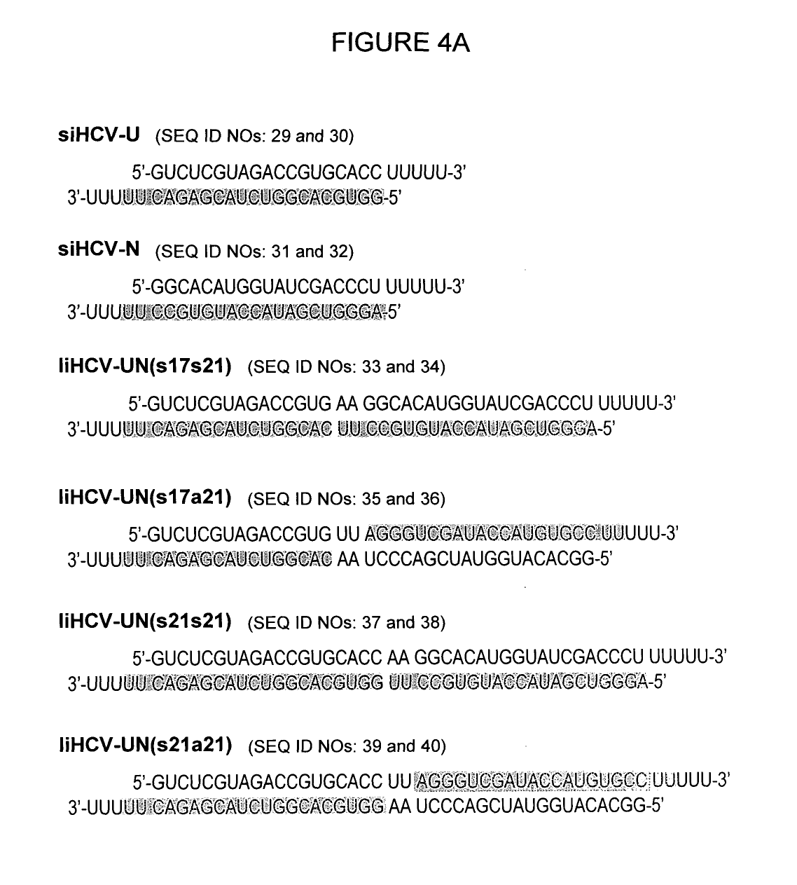

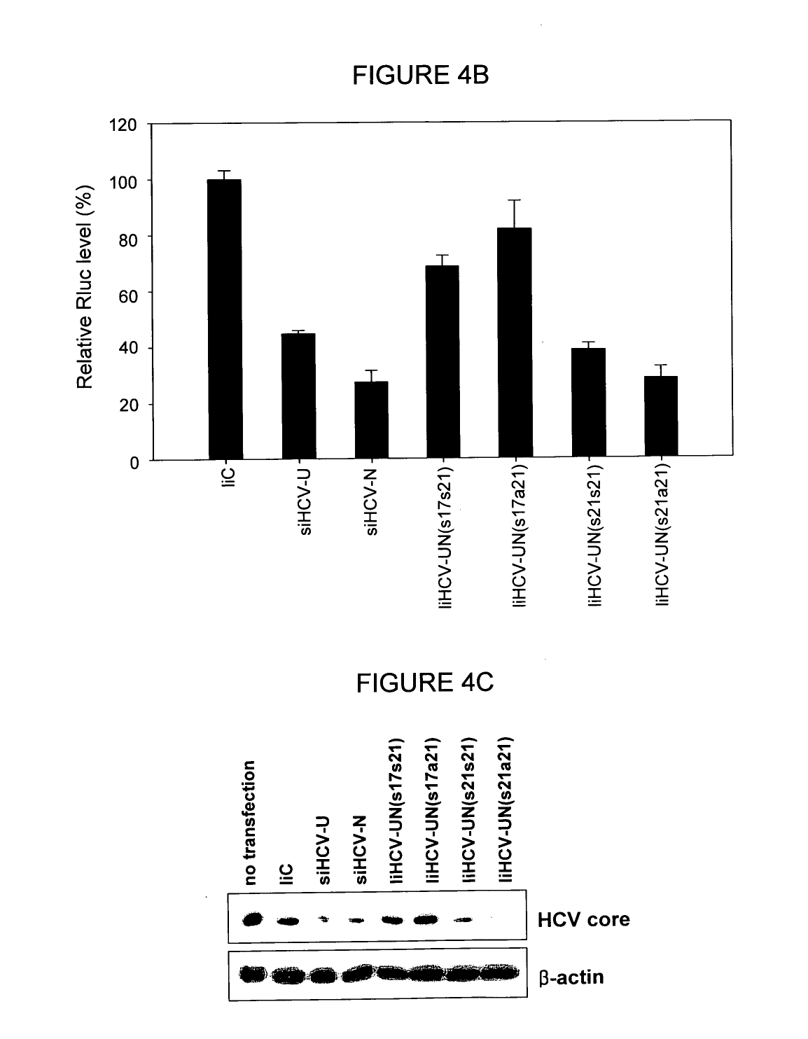

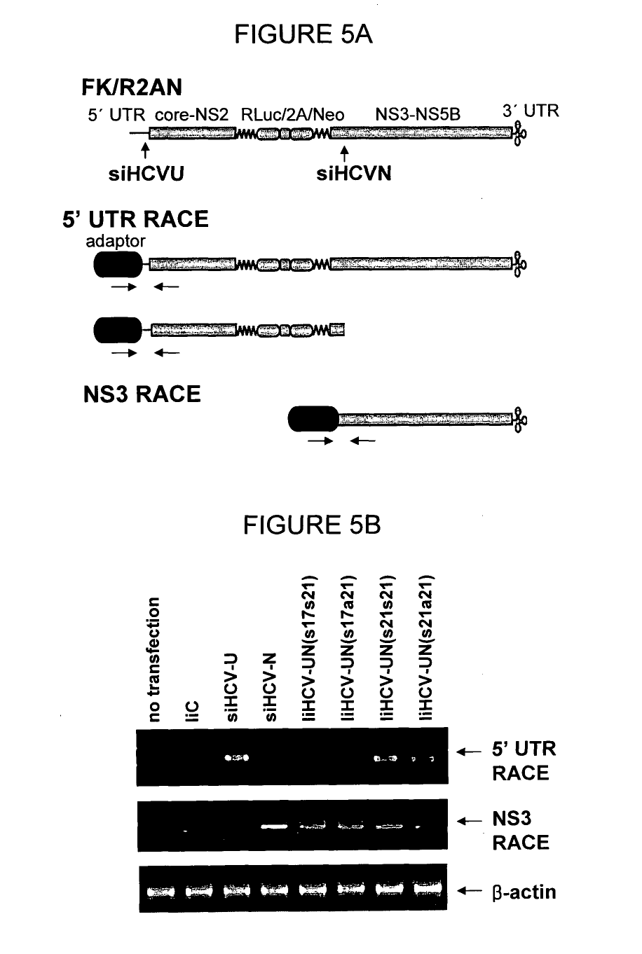

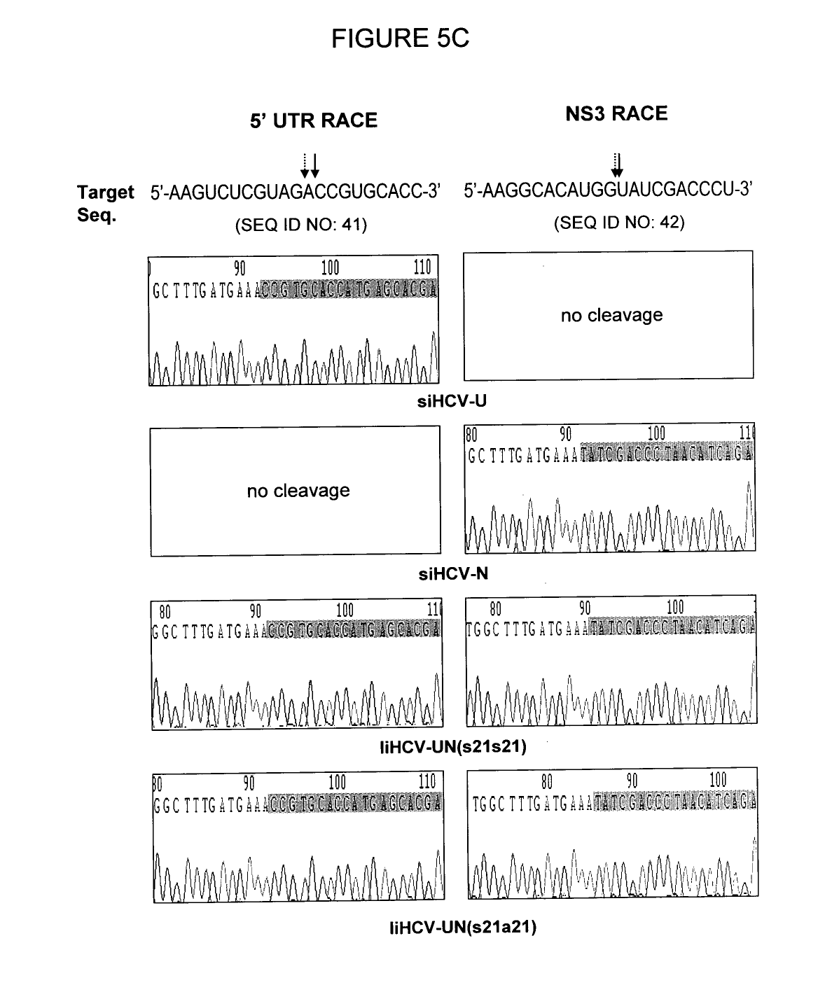

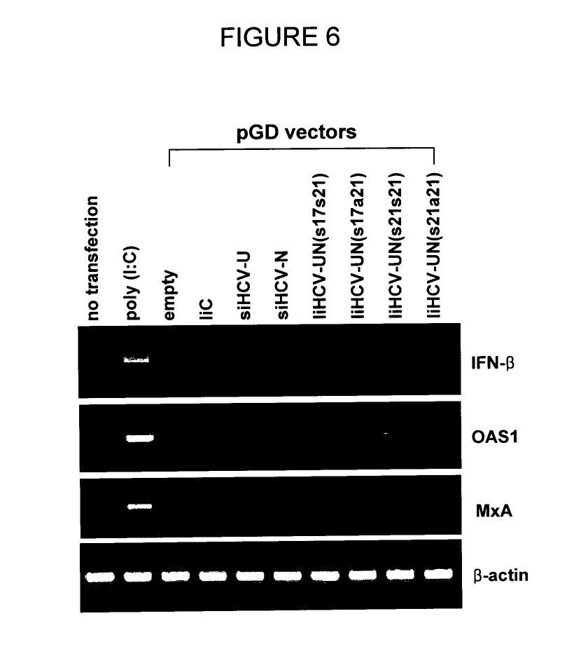

Furthermore, based on these results, multi-targeting activity of 40 by liRNA, 21 nt+21 nt in sense+sense and sense+antisense orientation, was confirmed in HCV replicon cells. Consistent with luciferase assay system, significant antiviral RNAi was induced by treatment with these liRNA-expression vectors as efficiently as each single siRNA-expression vector (see FIGS. 4B and 4C). Importantly, 5′ RACE and PCR data provide an evidence that both 5′ UTR and NS3 target sites simultaneously can be cleaved by liHCV-UN (s21s21) or liHCV-UN (s21a21) as specifically as by single siRNA (see FIGS. 5B and 5C). In addition, the inventive expression vector-derived liRNA did not induce non-specific IFN response in a Huh 7-based cell line, FK-R2AN, in which IFN activity is highly attenuated, indicating that the sequence-specific RNAi machinery but not the IFN response is a major factor for the anti-HCV activity in this study (see FIG. 6).

In summary, the present invention shows that different species of siRNA are processed from a vector-based, constitutively designed liRNA and then induce precise cleavage of target genes. The inventive liRNA may be applied for sporadic cleavage of viral RNAs with high mutation rate and for simultaneous knockdown of both pathogenic RNA and its host mRNA of functionally related proteins.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### Example 1

### Gene Silencing Activity of Double-Stranded RNAs Expressed from Two Convergent RNA Polymerase III Promoters

### (Step 1) Construction of DNA Plasmids

A dsRNA expression vector which contains two convergent RNA polymerase III promoters, human H1 and U6 promoters, and also termination signal between them was prepared as follows, according to previous reports (Shin D. et al., Virus Research 119:146-153 (2006)).

Specifically, a linear dsRNA expression cassette was prepared by PCR amplification of convergent human RNA polymerase III U6 and H1 promoters from pRNAiDu (Shin D. et al., supra) with their respective forward (f) primers, U6f, 5′-CGGAATTCCCCAGTGGAAAGAC-3′ (SEQ ID NO: 43), and H1f, 5′-CGGAATTCATATTTGCATGTCGC-3′ (SEQ ID NO: 44). The PCR product (˜470 bp) was purified from a 3% agarose gel and cloned into pGEM-T vector using the pGEM-T Easy Vector System (Promega, Madison, Wis.), and the resulting plasmid was named pGD-siC (FIG. 1A).

To test the ability of the dual promoter-based vector to express duplex siRNAs, the present inventors cloned synthetic oligonucleotides complementary to functionally validated EGFP- and RLuc-specific 21-mer siRNA sequences, i.e., siRNAs siE (s21) and siR (s21) shown in FIG. 1B, into the pGD-siC vector in place of the control DNA sequence, and named the resulting vectors pGD-siE (s21) and pGD-siR (s21), respectively. Other vectors containing the inverted correspondents of siE (s21) and siR (s21), named pGD-siE (a21) and pGD-siR (a21), were also prepared. In the resulting vectors, sense and antisense strands were transcribed from U6 and H1 promoter, respectively.

All sequences were verified using the BigDye Terminator Cycle Sequencing kit (ABI, Foster City, Calif., USA).

### (Step 2) Cell Culture and Transfection

Human hepatoma cell line Huh7 (ATCC CCL-185) and prostate cancer cell line PC-3 (ATCC CRL-1435) were maintained in Dulbecco's modified Eagle's medium (DMEM; HyClone, Logan, Utah, USA) supplemented with 10% fetal calf serum (FCS HyClone). At one day before transfection, Huh7 or PC-3 cells were seeded on 12-well plates at a density of 1.5×10⁵ or 2.0×10⁵ cells per well, respectively. To determine the gene silencing effect derived from siRNA- or liRNA-encoding plasmids, Huh7 cells were co-transfected with 10 ng of pEGFPLuc (encoding EGFP and FLuc fusion protein; BD Biosciences Clontech, Palo Alto, Calif., USA), 1 ng (of phRL-CMV (encoding RLuc protein; Promega) and 1 μg of a series of pGD using Lipofectamine 2000 (Invitrogen Carlsbad, Calif., USA) according to the manufacturer's instructions.

### (Step 3) Luciferase Assay

On days 1, 2 and 3, Huh7 cells transfected with pEGFPLuc (BD Biosciences Clontech) and phRL-CMV (Promega) as target plasmids in the presence of individual pGD vectors were lysed in 250 μl of 1×Passive Lysis Buffer (Promega) by shaking at room temperature for 30 min. Both firefly and Renilla luciferase expression levels were determined using Dual-Luciferase Reporter Assay System (Promega) according to the manufacturer's instructions.

Consequently, On day 2, significant gene knock-down by 80% on the average was observed with all encoded siRNAs, regardless of their DNA sequence orientation within the expression cassette, indicating that both RNA polymerase III promoters produce RNA molecules with similar transcription efficiency and that assembled linear duplex RNAs function in triggering RNAi (FIG. 1C).

### (Step 4) Gene Silencing Activity of Longer Duplex RNA Sequences

A long interference RNA (liRNA)-expression plasmid pGD-liER (s25s25) was constructed by fusion of the two 25-mer insert sequences, siE (s25) and siR (s25), which were extended to 3′ of their 21-mer target sites, eventually generating 48 by linear RNA duplex with 5 nt U sequences at the 3′ ends (FIG. 1B). Cell transfection and luciferase assay were carried out in accordance with the methods of Steps 2 and 3.

Interestingly, when pGD-liER (s25s25) was transfected, a dual luciferase assay revealed that EGFP-FLuc fusion expression was efficiently inhibited (FIG. 1D). In contrast, the target site against RLuc siRNA was only marginally suppressed following pGD-liER (s25s25) plasmid treatment. These data demonstrate that length or orientation of each siRNA component within the extended linear duplex RNA should be further optimized to induce precise cleavage of liRNA by Dicer, to enhance incorporation of potential antisense guide strand of processed siRNA into RISC and thus to more simultaneously and effectively reduce different target gene expression.

### Example 2

### Optimization of the Length of Long Interference RNA with Successively Connected Two siRNA Sequences

### (Step 1) Luciferase Assay

Because liER (s25s25) RNA has poor activity especially against RLuc gene as proved in Example 1, the length of siRNA sequences corresponding to RLuc was reduced to 21-mer by cloning of pliER (s25s21) (FIG. 2A). After cotransfection of this plasmid with target vectors into Huh7 cells, pEGFPLuc and pCMV-hRL, dual gene knockdown ability was measured by luciferase assay on day 2, in accordance with the methods of Steps 2 and 3 of Example 1. Interestingly, as shown in FIG. 2B, EGFPLuc gene was reduced 60%, whereas inhibition of RLuc expression (20% knockdown) was still marginal. Thus, 25 nt siE RNA sequence was further reduced to 21 and 15 nt, generating pliER (s21s21) and pliER (s15s21), respectively (FIG. 2A). Both FLuc and RLuc expression was most efficiency silenced by pliER (s21s21). When shortening the siE sequence to 15 nt, EGFP gene silencing absolutely disappeared although it has RNAi activity against RLuc gene (FIG. 2B).

### (Step 2) Northern Blot Analysis

In order to compare the processing efficiency of liRNAs into correct siRNA products in culture cells, a northern blot analysis was carried out as follows.

Total RNAs were extracted from Huh7 cells transfected with pGD vectors (pGD-liER(s25s21), pGD-liER (s21s21), pGD-liER (s15s21) and liC) on day 2 using Trizol reagent (Invitrogen). Five micrograms of total RNA per lane were loaded on 7 M urea/15% polyacrylamide gel and transferred to a Hybond N⁺ nylon membrane (Amersham Bioscience, Piscataway, N.J.). The membrane was hybridized with [γ−³²P]-labeled DNA probes complementary to siRNA antisense strand or U6 small nuclear RNA in ExpressHyb Hybridization Solution (Clontech Laboratories, Inc., Mountain View, Calif., USA) according to the manufacturer's protocol. Probe sequences were as follows: EGFP siRNA probe, 5′-GCAGCACGACTTCTTCAAG-3′(SEQ ID NO: 45); RLuc siRNA probe, 5′-GGGCGAGGTTAGACGGCCT-3′ (SEQ ID NO: 46); and U6 RNA probe, 5′-TAGTATATGTGCTGCCGAAGCGAGCA-3′ (SEQ ID NO: 47). The radiolabeled Decade Marker System (Ambion, Austin, Tex. USA) was used as an RNA marker.

The Northern blot data with each siE and siR probe, which hybridizes their antisense strand, revealed that both siE and siR product of 21-22 nt was produced only in pliER (s21s21)-transfected cells (FIG. 2C). However, partial or inefficient siRNA production was detected by pliER (s25s21) and pliER (s15s21). This result was well correlated with the luciferase assay (FIG. 2B). These results demonstrate that the most optimal length of siRNA unit successively connected within liRNA is 21 nt in respect of RNAi activity and siRNA generation in mammalian cells.

### Example 3

### Optimization of the Length of liRNA with Convergently Connected Two siRNA Sequences

Most siRNA design programs by the use of RNA pol III-derived expression cassette prefer purine sequence as an initiation nucleotide. However, two siRNAs are linked successively within liRNA expression vector, the first nucleotide of the antisense sequence could be the pyrimidine sequence, possibly lowering the transcription efficiency of that strand relative to its sense strand. This can make a limitation for the preparation of liRNA expression vectors with high transcription efficiency. Accordingly, to provide the wide applicability of linear liRNA, we prepared liRNA expression series (liER (s25a21), liER (s15a21)) and liER (s25a21)) that containing two convergently connected siRNAs with different lengths (FIG. 3A). Their ability to inhibit both EGFPLuc and RLuc gene expression was assessed by dual luciferase assay as in Step 3 of Example 1 after cotransfection with target DNAs into Huh7 cells in accordance with the method of Step 2 of Example 1.

Notably, simultaneous gene silencing was achieved by the use of pliER (s21a21), while only partial or insignificant activity was resulted from pliER (s25a21) and pliER (s15a21)(FIG. 3B). Consistent with this observation, guide strand of two functional siE and siR products with correct size (21-22 nt) was detected in pliER (s21a21)-transfected cells (FIG. 3C). The data indicate that alignment of two siRNAs of 21 nt to be transcribed from each RNA pol III promoter is also functioned as efficiently as consecutively linked one.

### Example 4

### Inhibition of HCV Replication by Long Interference RNA

### (Step 1) Preparation of HCV Replicon Cell Line

HCV genotype 1b replicon cell line FK/R2AN was constructed by insertion of the fused Renilla luciferase and FMDV 2A genes upstream of the neomycine resistance cassette within the original R2AN replicon (Genbank Assession No: AJ238799). Cells were maintained in DMEM (HyClone) containing 10% FBS (HyClone) and 0.6 mg/ml G418 (Calbiochem, La Jolla, Calif., USA). Before transfection, viral replication in replicon cells was validated by luciferase assay, Western blot analysis or real-time RT-PCR as described previously. To test RNAi activity of expressed siRNA or liRNA, 2.0×10⁵ cells were seeded in wells of 12-well plates, transfected with 1 μg of pGD-derived plasmids using Lipofectamine 2000 (Invitrogen) and then incubated for 2 days at 37° C.

### (Step 2) Luciferase Assay

Plasmids expressing HCV-specific siRNA, siHCV-U, targeting 21 nt of HCV 5′ UTR and siHCV-N targeting 21 nt of HCV NS3, were prepared by cloning into the pGD vector backbone (FIG. 4A). These plasmid-derived siRNAs reduced Renilla luciferase expression level by 60 and 80%, respectively, in full-length HCV replicon cells FK/R2AN on day 2 post-transfection relative to irrelevant siRNA or liRNA (liC) (FIG. 4B). In replicon cells, the luciferase reflects relative viral replication or gene expression level as a reporter.

Additionally, dual siRNA vectors of different lengths containing siHCV-U and siHCV-N target sequences were constructed and alignments, expressing liHCV-UN (s17s21), liHCV-UN (s17a21), liHCV-UN (s21s21), and liHCV-UN (s21a21) (FIG. 4A). Luciferase assay shows that the latter two 21+21 constructs inhibited HCV replication efficiently in vitro, whereas the former two, liHCV-UN (s17s21) and liHCV-UN (s17a21), was not so potent when compared with single siRNAs (FIG. 4B).

### (Step 3) Westeren Blot Analysis

To measure HCV protein expression, FK/R2AN recplicon cells alone or transfected with pGD vectors were lysed using M-Per Mammalian Protein Extraction Reagent (Pierce, Rockford, Ill., USA). Total cell lysates (30 μg) were resolved in a 12% SDS polyacrylamide gel and transferred to the Immobilon-P PVDF membrane (Millipore, Bedford, Mass., USA). HCV core was identified using their specific primary antibodies purchased from Affinity Bioreagents (Golden, Colo. USA). Cellular β-actin protein was probed using anti-human β-actin antibody (Sigma, St Louis, Mo. USA). The band intensities were quantified with ImageJ public domain software from the National Institutes of Health of the U.S.A.

Consistent with the result of luciferase analysis, viral core protein expression was dramatically reduced by 21+21 liRNAs but not by 17+21 liRNA (FIG. 4C). These results proves that the length of each target sequences within the extended multi-functional duplex RNAs is 21-mer and, in this case, their orientation is not a critical factor. Moreover, it should be stressed that reduction of nucleotide number of only one target can affect the rest 21-mer siRNA's RNAi activity and overall antiviral potency in a cis target expression system.

### Example 5

### Identification of Cleavage Sites within in HCV RNA by liRNAs

To evaluate the viral RNA cleavage by expressed liRNAs and to determine 5′-ends of its cleavage sites, each 1.5 μg of total RNAs from FK/R2AN replicon cells transfected with siRNA- or liRNA-expression vector was ligated with a 5′ RACE RNA Adapter using FirstChoice RLM-RACE Kit (Ambion) according to the manufacturer's instructions. They were amplified with two sets of outer primers as follows: for primary amplification of 5′ UTR, 5′ RACE Outer Primer from the Ambion's RACE kit and 5′ UTR outer primer, 5′-ACTAGGCCGAGAGCCACGGG-3′ (SEQ ID NO: 48); and, for primary amplification of NS3, the same 5′ RACE Outer Primer and NS3 outer primer, 5′-TTGGTCCAGGACTGTGCCGAT-3′ (SEQ ID NO: 49). Then the DNA was reamplified with two sets of inner primers as follows: for secondary amplification of 5′UTR, 5′ RACE Inner Primer from the Ambion's RACE kit and 5′ UTR inner primer, 5′-TCCACGAGGTTGCGACCGCT-3′ (SEQ ID NO: 50) for secondary amplification of NS3, the same 5′ RACE Inner Primer and NS3 inner primer, 5′-GTCAGTTGAGTGGCACTCAT-3′ (SEQ ID NO: 51). The amplified products were resolved on a 3% agarose gel, and bands for 5′ UTR (220 bp) and NS3 (177 bp) were eluted, separately. Following cloning of the amplified DNA into pGEM-T vector (Promega), their cleavage sites were determined by sequencing analysis.

As shown in gel electrophoresis of PCR products, plasmid-derived expression of siHCV-U and siHCV-N in replicon cells created their own PCR products (FIG. 5B). Any amplified PCR band with corresponding sizes was not detected in non-transfected or control liC-expression plasmid-treated cells. Notably, both 5′ UTR and NS3 RACE products were detected in liHCV-UN (s21s21) and liHCV-UN (s21a21) RNA-expressing cells. However, only NS3 RACE-derived PCR amplification was achieved by liHCV-UN (s17s21) and liHCV-UN (s17a21), indicating that these two liRNAs can be processed into functional siHCV-N but not siHCV-U.

Furthermore, to determine the cleavage sites within viral RNA, the individual above PCR products were directly inserted in pGEM-T vector. The sequencing analyses shows that siHCV-U cleaves HCV 5′ UTR at the one base-shifted site to the 3′-terminus (between the nucleotides paired to bases 9 and 10 of the antisense siRNA) mainly than the expected site (between the nucleotides paired to bases 10 and 11 of the antisense siRNA), while siHCV-N recognize the expected cleavage site within the NS3-coding region (FIG. 5C). Importantly, these sequencing data clearly provide evidence that 21+21 liRNAs, liHCV-UN (s21s21) and liHCV-UN (s21a21), are be able to target and cut both 5′ UTR and NS3 sites as correctly as single siRNA. Taken together, the results support that optimal length of siRNA units within liRNA is about 21-mer to generate functional siRNA components and to knockdown simultaneously different targets by cellular RNAi machinery.

### Example 6

### Interferon Response to liRNAs in Mammalian Cells

It has been reported that dsRNA longer than 30 by can induce non-specific gene silencing mediated by activation of IFN signaling pathway. However, Huh 7 is a representative mammalian cell line with tolerance to this dsRNA-involved IFN stimulation. To determine whether observed anti-HCV effect of liRNAs was even partly due to induction of interferon response by long dsRNA, mRNA expression levels of IFN-β, OAS1, and MxA were measured by semi-quantitative RT-PCR after transfection of each siRNA or liRNA expression plasmid into FK/R2AN cells.

Specifically, total RNA isolated from FK/R2AN cells was reverse transcribed into cDNA on day 2 after transfection with 1 mg of synthetic siRNA or different pGD vectors. Poly(I):poly(C) dsRNA was used as a positive control to induce the IFN response. Human IFN-β, OAS and MxA cDNAs were amplified with their respective primers. The forward and reverse primers used are as follows: for IFN-β gene, 5′-ATGACCAACAAGTGTCTCCT-3′ (SEQ ID NO: 52) and 5′-TCAGTTTCGGAGGTAACCTG-3′ (SEQ ID NO: 53); for OAS gene, 5′-TCAGAAGAGAAGCCAACGTGA-3′ (SEQ ID NO: 54) and 5′-CGGAGACAGCGAGGGTAAAT-3′ (SEQ ID NO: 55); and, for MxA gene, 5′-AGTATGGTGTCGACATACCGGA-3′ (SEQ ID NO: 56) and 5′-GAGTCTGGTAAACAGCCGAATG-3′ (SEQ ID NO: 57). The PCR products were analyzed on a 2% agarose gel. In parallel, 18S and 28S RNA bands were visualized following electrophoresis of total RNA and ethidium bromide-staining to ensure equal sample amounts.

As shown in FIG. 6, neither 21-mer siRNA nor liRNA (17+21 or 21+21) did activate the IFN pathway except for high stimulant poly(I:C), indicating that above in vitro gene silencing activity against HCV was from sequence-specific RNAi activity. Interestingly, in cells transfected with plasmids expressing 17+21 liRNAs such as liHCV-UN (s17s21) and liHCV-UN (s17a21), IFN activation was not significant as in pGD-empty, -siHCV-U, or -siHCV-N treated cells. These observations indicate that 21+21 HCV-specific liRNA with dual-targeting RNAi potency in a sequence specific manner can also stimulate IFN response in a sequence-independent manner.

While the invention has been described with respect to the above specific embodiments, it should be recognized that various modifications and changes may be made to the invention by those skilled in the art which also fall within the scope of the invention as defined by the appended claims.

What is claimed is:

1. A linear double-stranded RNA molecule which comprises two to ten consecutively or convergently linked short interfering RNAs (siRNAs) each reducing the expression of one of different target genes.

2. The linear double-stranded RNA molecule of claim 1, wherein each of the siRNAs comprises 18 to 24 nucleotides.

3. The linear double-stranded RNA molecule of claim 1, wherein each of the siRNAs comprises 21 nucleotides.

4. The linear double-stranded RNA molecule of claim 1, which consist of two consecutive or convergent siRNAs, 40 nucleotides of one strand of the RNA molecule being base-paired with the complementary 40 nucleotides of the other strand and each strand comprising two 3′ terminal nucleotides that are not base-paired.

5. The linear double-stranded RNA molecule of claim 1, wherein the target genes are viral genes.

6. A recombinant expression vector comprising double-stranded DNA sequences expressing the linear double-stranded RNA molecule of claim 1.

7. The recombinant expression vector of claim 6, which comprises two convergent promoters operably linked to both ends of the doublestranded DNA, each promoter allowing transcription of each strand of the double-stranded DNA sequence.

8. The recombinant expression vector of claim 6, wherein the promoters are human RNA polymerase III promoters each of which is contiguous with each of 5′ ends of the double-stranded DNA.

9. The recombinant expression vector of claim 8, wherein the human RNA polymerase III promoters are selected from the group consisting of human HI, U6, 5S rRNA, 7SK and tRNA promoters.

10. The recombinant expression vector of claim 6, wherein each of the siRNAs contained in the linear double-stranded RNA molecule comprises 18 to 24 nucleotides.

11. The recombinant expression vector of claim 6, wherein each of the siRNAs contained in the linear double-stranded RNA molecule comprises 21 nucleotides.

12. The recombinant expression vector of claim 6, wherein the linear double-stranded RNA molecule consist of two consecutive or convergent siRNAs, 40 nucleotides of one strand being base-paired with the complementary 40 nucleotides of the other strand and each strand of the RNA molecule comprising two 3′ terminal nucleotides that are not basepaired.

13. A composition comprising the linear double-stranded RNA RNA molecule of claim 1.

14. A method of reducing expression of target genes in a cell, the method comprising introducing the linear double-stranded RNA molecule of claim 1 into the cell, wherein the encoded siRNAs target different genes and reduce expression of the target genes.

15. The method of claim 14, wherein the cell is a eukaryotic cell.

16. The method of claim 14, wherein the cell is in vitro.

17. The method of claim 14, wherein the target genes are endogenous genes.

18. The method of claim 14, wherein the target genes are exogenous genes.

19. The method of claim 18, wherein the target genes are genes of an intracellular pathogen.

20. The method of claim 19, wherein the target genes are viral genes.

21. The method of claim 20, wherein the viral genes are hepatitis C virus (HCV) genes.

22. The method of claim 21, wherein the linear double-stranded RNA molecule is composed of a set of nucleotide sequences of SEQ ID NOs: 37 and 38 or SEQ ID NOs: 39 and 40.

23. A composition comprising the recombinant expression vector of claim 6.

24. A method of reducing expression of target genes in a cell, the method comprising introducing the recombinant expression vector of claim 6 into the cell, wherein the encoded siRNAs target different genes and reduce expression of the target genes.
